(19) United States
(12) Patent Application Publication
(10) Pub. No.: US 2009/0180921 A1
(43) Pub. Date: Jul. 16, 2009

(54) Method of collecting, processing, sterilizing and stabilizing rumen fluid and the contained bacterial polysaccharides

(76) Inventor: Robert Terrell Winn, Big Sandy, TX (US)

Correspondence Address: Robert Terrell Winn, 141 PR 3808, Big Sandy, TX, 75755, US

(21) Appl. No.: 12/217,553

(22) Filed: Jul. 8, 2008

Related U.S. Application Data
(62) Division of application No. 10/923,313, filed on Aug. 23, 2004, now abandoned.

(51) Int. Cl. A61L 2/00

(52) U.S. Cl. 422/1

(57) ABSTRACT

A method of collecting, processing, sterilizing, and stabilizing rumen fluid to retain the bioactivity of the bacterial polysaccharides found therein is described. The feeding of this resulting composition the first few days of young animals' lives aids in decreasing diarrhea morbidity, severity and mortality. In addition it results in increased growth rate.

### CROSS-REFERENCES

This application is a Divisional Application of application Ser. No. 10/923,313, filed on Aug. 23, 2004, entitled “Animal Nutritional Product that Increases Weight Gain and Reduces Diarrhea Morbidity, Mortality and Severity by Stimulation of Natural Immune Response, Nutritional Support of Immune Function and Supplemental Nutricines and Probiotics.”

### FEDERALLY SPONSORED RESEARCH OR DEVELOPMENT

The development and research for this invention involved no federal or state funding. It was supported in full by private funding.

### COMPACT DISCS AND ELECTRONIC DATA

There are no electronic data or compact discs included with this submission.

### DETAILED DESCRIPTION AND SPECIFICATION

### Field of the Invention

The present invention relates to the collection, processing, sterilization and stabilizing of rumen fluid in a dry form to preserve and standardize the bacterial polysaccharides contained therein. It further relates to the subsequent utilization of this product with a mixture of probiotics, nutricines, vitamins, minerals, an amino acid, and a monosaccharide. In particular this invention is fed to young animals for the first few days of life to increase weight gain, reduce diarrhea severity, morbidity and mortality by stimulation and support of the animals natural immune response.

### BACKGROUND OF THE INVENTION

Animals are raised in concentrated rearing units. These units are used on a constant basis resulting in a build up of contamination and disease organisms. The young newborn animals are frequently affected with diarrhea. Although management practices to maximize the passive immunity are used and sanitation measures followed to minimize the exposure of newborns to virulent organisms, the diarrheal disease process is the most costly disease process affecting the rearing of newborns.

There is both a political move and a public health concern with the use of antibiotics as feed additives. There are also public health concerns with the extra-label use of antibiotics in food producing animals. To maintain health and increase productivity without the use of antibiotics is the goal of many endeavors at this time (Donovan, D. C., et al, Growth and Health of Holstein Calves Fed Milk Replacers Supplemented with Antibiotics or Enteroguard, 2002, J. Dairy Sci. 85:947-950: Webb, P. R., et al, Addition of fructooligosaccharide (FOS) and sodium diacetate (SD) plus decoquinate (D) to milk replacer and starter grain fed to Holstein calves, 1992, J. Dairy Sci. Vol 75 Suppl. 1:300). As such, there are many studies and products, which attempt to increase the immuno-competence of the neonate. Vaccines, serum immunoglobulins, colostrum replacers and colostrum antibody preparations have all been used to improve the neonate's immune status. Other nutritional supplements have been described. U.S. Pat. No. 6,667,063 B2 describes a composition containing as the essential ingredients colostrum, a selected whey product and defined amounts of selenium or an organic or inorganic, water soluble selenium precursor. The goal, ingredients and method of action are different from the present invention.

U.S. Pat. No. 5,374,425 describes the manufacture of a killed probiotic. The stabilization process is somewhat similar to the process used in the current invention. Both products are autoclaved to kill the bacterial cells. In the current invention, autoclaving takes place at 116° C. for 45-60 minutes at a pressure of 10 p.s.i. The referenced patent uses a variable temperature (100° to 121° C.) and a shorter duration (15-30 minutes). Also there is also a difference in drying. To separate the bacteria cells in U.S. Pat. No. 5,374,425 a flocculating agent is added to the culture and the cells are allowed to settle out. The liquid is decanted off. Heat, spray or freeze-drying is promoted as acceptable drying methods and the use of a drying agent is proposed. These methods except for freeze-drying are not acceptable in the current invention. Another difference is that the current patent uses rumen fluid bacteria, while this patent uses a specific culture or mixes of dried specific cultures. U.S. Pat. No. 4,021,303 also produces killed organisms. This process includes chemically treating the microorganisms with alkali at a pH of 10.5-12.9 and a temperature of 0°-30° C., washing with water and mechanically rupturing the bacteria at a pH of 7-10.2.

Another aim of this invention is to increase the natural local immune response by the exposure of the gut to bacterial polysaccharides in a measured, safe and controlled manner. Rumen fluid has been shown to increase growth rate in calves, decrease morbidity, mortality and use of treatments for diarrheal disease (Muscato, T. V., L. O. Tedeschi, and J. B. Russell, The Effect of Ruminal Fluid Preparations on the Growth and Health of Newborn, Milk-Fed Dairy Calves, 2002, J. Dairy Sci., 85:648-656). Rumen fluid has been shown to contain bacterial polysaccharides. These bacterial polysaccharides are considered the “active ingredient” in rumen fluid. Bacterial polysaccharides have been shown to elicit localized immunity. Rumen bacteria have been reported to have extracellular polysaccharide “coats” that are similar to those found on many Gram (−) organisms (Costerton, J. W., H. N. Damgaard and J. K. Cheng, Cell envelope morphology of rumen bacteria, 1974, J. of Bacteriology, 118:1132-1143). It is my belief that this similarity is the reason ruminal fluid bacteria are the best to use for this desired result.

We are taught in U.S. Pat. No. 6,444,210 B1 that bacterial polysaccharides have been used as vaccines to enhance specific humoral immunity and in the particular invention named they are used to enhance general cellular immunity against a wide variety of microorganisms. The mentioned patent describes a method of isolation, purification, stabilizing and using Brucella abortus and Yersinia enterocolitica outer polysaccharide as an immunizing agent. This differs from the current invention in that the current invention makes no strides toward selecting, isolating or purifying a particular polysaccharide considered effective as an immune modulator. It further differs from the current invention in that the current invention makes no effort toward selecting, isolating or purifying the bacterial polysaccharide from the rest of the ingredients in the rumen fluid, except for excluding physically large fibers and particles. Also, the number of species of bacteria in the rumen is great and there are no steps taken to reduce this number of species. Three other similar claims have been made for specific extracts of polysaccharides to be used as vaccinal agents, see U.S. Pat. Nos. 4,210,641; 6,007,818; and 6,045,805. The current invention differs from these three inventions for the aforementioned reasons.

We are told in U.S. Pat. No. 6,087,342 that the extraction of polysaccharides that have immune stimulating properties results in small fragments of the longer chain immune-stimulating polysaccharides. These fragments that occur have lower bioactivity than that found in the parent substance. This patent involves the use of a special substrate to bind the small fragments to which potentates the activity of the fragments. This differs from the current invention in two main aspects. First an isolated product in the form of bacterial polysaccharides or bacterial nucleic acids from bacteria is used. Second this is bound to a specialized substrate. My invention uses the whole rumen fluid, or the whole bacterial culture, as it were. I also use the rumen ingesta smaller than 2 mm as the substrate that is used to carry the bacteria.

Another novel method of stimulating the immune system with bacterial produced products is described in U.S. Pat. No. 5,840,318. This method consists of growing bacteria in a stressed manner to increase the stress response factors production of the bacteria. These products are then isolated and used to activate and modulate circulating macrophages. This differs from the current invention in several methods, but primarily due to the fact that the bacteria are stressed instead of grown to peak growth rates. The stress response factors desired by the described method are not a consideration in the current invention.

Bacterial polysaccharides are produced under several patents for use as food thickeners. These patents use bacteria of the genus Xanthomonas and describe a process to grow the bacteria using specialized media or growing conditions. These descriptions are found in U.S. Pat. Nos. 3,328,262; 3,391,061; 3,433,708; and 4,692,408. Other bacterial polysaccharides are produced for use as viscosity regulators used in various manufacturing processes as described in U.S. Pat. No. 4,567,140.

Rumen fluid fed fresh has resulted in increased growth rate in calves, decreased morbidity, mortality and use of treatments for diarrheal disease (Muscato, T. V., L. O. Tedeschi, and J. B. Russell, The Effect of Ruminal Fluid Preparations on the Growth and Health of Newborn, Milk-Fed Dairy Calves, 2002, J. Dairy Sci., 85:648-656). The obvious problems to using fresh rumen fluid are the daily collection of the fluid. The chance of spreading disease. The need to maintain a fistulated animal on each farm. Rumen fluid may be sterilized and bottled to increase storage time. However, upon opening, the bottle must be refrigerated. Also, each farm would need to maintain the equipment to sterilize the rumen fluid.

The process of the current invention allows for the collection of rumen fluid; sterilization of the fluid to prevent disease spread and stabilizing the rumen fluid to retain the bioactivity of the bacterial polysaccharides found therein.

U.S. Pat. No. 4,228,275 describes a process of producing a nitrogen containing polysaccharide. This process includes the use of a specific bacterium, not many species of bacteria as is found in the rumen. It also requires reaction with and aqueous ammoniacal solution at a temperature of 100° to 250° C. The resulting product is used to control viruses in plants. U.S. Pat. No. 4,529,701 describes a method of stimulating bacterial growth in an anaerobic digestion system. It specifically deals with improving digestion in sewage digestion systems that have gone sour and uses a mixture of an inhibitory ion regulation component and an inorganic pyrophosphate-containing compound.

This is not the first process to take advantage of products produced by microorganisms. I would draw your attention to some patented processes that may on first glance appear similar to this process. In U.S. Pat. No. 6,255,080 B1 rumen bacteria of the Butyrivibrio spp. are used to produce proteinaceous antibiotics that are resistant to gastric proteases, exhibit a high level of hydrophobicity, and are effective under anaerobic conditions. The Butyrivibrio spp. are isolated and cultured and screened for their production of bacteriocin-like activity. In U.S. Pat. No. 1,818,781 mixed cultures of bacteria were used to cause specialized fermentation to produce ethyl alcohol, lactic acid, butyric acid, butyl alcohol, isopropyl alcohol, acetone, etc. Neither patent uses the same growth media, apparatus nor obtain the same end product as the current application.

An important goal of this invention is to make the product available in an easily storable, transportable and usable form. Dried rumen fluid was considered not to have the activity of liquid rumen fluid and therefore not to be a viable alternative (Dr. J. Russell, Personal communication, Aug. 6, 2002). Field trials by the inventor using warm forced air-dried rumen fluid on drying aids have not to given the beneficial results obtained with sterilized liquid rumen fluid. Field trials by the inventor with freeze dried sterilized rumen fluid have been shown to give results equal to those obtained with sterilized liquid rumen fluid. This gives this invention the distinct advantages of being easily stored, transported, used and standardized for bacterial polysaccharides. In U.S. Pat. Nos. 4,855,149 and 4,877,634 a method of drying bacterial polysaccharides produced from a culture of Leuconostoc is described. The process includes using drying aids and preferably spray drying or drying “in any manner”. The end product is to be used as quality improvers (e.g. texture, stability or thickness) for foods. U.S. Pat. No. 4,391,887 describes a method of drying mixed cultures of bacteria to maintain the bacterial activity. This process differs in that live cultures are stored to be used as inoculates for the degradation of industrial organic effluents.

This product may be administered orally to individual animals by either drenching or dosing with a solution of the product. It may be fed to individual animals by mixing it into the milk fed to that animal. It may be fed by some unique system as mentioned above for poultry. Or, it may be top-dressed on dry feed for swine and poultry. The feeding period will range from 3-7 days and should start on day 1 or 2 of life.

### SUMMARY OF THE INVENTION

A process of collecting, processing, stabilizing and drying rumen bacterial polysaccharides. These dried rumen bacterial polysaccharides maybe included into a novel composition with vitamins, probiotics, minerals, an amino acid and monosaccharide are used to reduce the effect of diarrhea in young animals. The present invention retains the bioactivity of the polysaccharides, while other methodology does not.

### DRAWINGS

There are no drawings.

### SPECIFICATION

The invention A Method of Collecting, Processing, Sterilizing and Stabilizing Rumen Fluid and the Contained Bacterial Polysaccharides is actually a method. The first section will be the process by which rumen fluid is harvested to protect and preserve the bacterial polysaccharides within it, in a manner that is easily mixed, standardized, stored, transported, measured and re-hydrated.

### Process

The animals used for this process must be selected to be of sufficient size to support removal of rumen fluid from a rumen cannula that is placed into surgically created rumen fistulas in their left flank region. This is also important in that the size of cannula needed for daily withdrawals of fluid is a four-inch (4″) cannula. If a smaller animal is used, the fistula and the cannula may be below the fluid line in the rumen. This would allow for constant seepage of rumen fluid and irritation to the area of the rumen fistula. This would severely limit the length of time of collection that would be possible in the donor animal. This would also drastically affect the health of the fistulated animals. The animals should be of a temperament to allow daily man to animal contact, manipulation, handling and care with the least of actual physical restraint.

The animals should be tested for Johne's disease, TB, and BVD. Brucellosis testing or vaccination is recommended. Vaccination with IBR, PI3, BVD and BRSV are recommended. Also the Clostridial diseases and Leptospirosis should be included in the vaccination program. Parasite control should be administered as needed.

The surgical preparation and placement of the rumen fistula should be done a minimum of three (3) weeks prior to actual time of onset of collection. This will allow for healing of the incision site, removal of the sutures, and placement of the final cannula for long-term care and collection. The site for fistula insertion should be high on the left flank, but not so high that the cannula will be above the transverse processes on the outside of the animal, nor be forced to bend under the processes on the inside of the rumen. This will allow for a minimum of irritation from the cannula itself. This is paramount in keeping the donor animal healthy. As mentioned above, the bottom of the fistula should be above the fluid line in the rumen to prevent constant seepage. The surgical fistula should be round, that is, the same shape of the cannula. It should be four-inches (4″) in diameter when cut. A smaller cannula (three-inch, 3″) should be placed into the fistula following surgery to allow healing. This can be replaced at two to three weeks following surgery and removal of the sutures. The placement of the final four-inch (4″) cannula should not be done until the entire suture line is healed. If a smaller cannula is not placed into the fistula, the fistula will contract during the healing process and will prevent the use of the larger permanent cannula later (info@bardiamond.com, Cannulae Surgery Information; P. L. Rohwer, Personal communication, Jan. 2, 2003).

The animal should be placed on a special diet to increase the level of rumen bacteria. This should be done a minimum of two (2) weeks prior to the actual time of onset of rumen fluid collection. This portion of the process will allow the rumen bacteria to adjust to the new feed and increase in numbers and growth rate. This will also allow the rumen contents to gain the actual texture necessary to allow fluid collection without having to either remove dry ingesta, add water to the rumen fluid or remove the ingesta and rinse it with water to “wash out” the bacteria. The diet formulation, physical size and feeding regularity are all important in regulation of the rumen mat, which in turn affects the ease of collection of rumen fluid and the growth of bacteria.

Feeding should be done a minimum of two times a day. The timing of the feedings should be such that the major feeding period is done 12 hours prior to collection and a secondary feeding with grain done 4-8 hours prior to collection. The feedings should be coincided such that the collections may be made between 1100 and 1500 hours. This timing results in a near peak production of rumen bacteria (Bryant, M. P., and I. M. Robinson, Effects of Diet, Time After Feeding and Position Sampled on Numbers of Viable Bacteria in the Bovine Rumen, 1968, J. Dairy Sci., 51:1950-1955; Bryant, M. P., and I. M. Robinson, An Improved Nonselective Culture Media for Ruminal Bacteria and its use in Determining Diumal Variation in Numbers of Bacteria in the Rumen, 1961, J. Dairy Sci., 44:1446-1456). Multiple feedings keep the bacterial mass growing and near the peak concentration, thereby allowing for larger collections of bacterial polysaccharides. Multiple feedings also prevent large swings in bacterial growth that might influence the types of bacterial available. It is important that multiple species are available for collection to give the widest range of possible bacterial polysaccharide attachments for the neonate's gut to recognize and to subsequently develop immunity against.

The total amount collected is approximately one to one-and-one/half (1-1½) gallons of rumen fluid per collection period. A collection period is defined as the actual collection of fluid ingesta by withdrawal of fluid through a collection tube. The collection is made by first removing the cap on the rumen cannula. The hose is introduced into the rumen by first “clearing” a path with a sleeved arm. This accomplishes two goals. One is to allow the passage of the collecting hose through the rumen mat so that it may reach the fluid level. The second is to free up liquid in the mat that contains a higher level of bacteria. The hose end is covered with your hand when introducing into the rumen down to the level of the rumen fluid. The hose used may be solid or have small holes in the wall in the last six-inches (6″) of the inserted end. Vacuum or siphoning is then used to withdraw the fluid.

A maximum of one collection period per day is performed. This allows the donor animal time to readjust her rumen fluid level, consume more feed to replenish what has been removed, and to readjust her electrolytes that may be affected by removing rumen fluid without the lower gut having the chance to regain nutrients that are contained within.

Once the fluid is removed, it should be collected into a stainless steel, glass or specially designed hard plastic receptacle to prevent any reaction between the fluid and the receptacle. The receptacle should be clean, disinfected or sterilized and rinsed with de-ionized or distilled water. This physical composition requirement and cleaning methodology will be the same for all of the numerous processing receptacles and utensils.

The fluid is then sieved through a series of sieves, starting with the largest size holes first and progressing to the smallest. The final size sieve should have holes a maximum of two (2) millimeters in diameter. The final solution will contain a slight amount of sediment. The solution should be mixed thoroughly enough to suspend this sediment and then metered into containers for autoclaving. The mixing process must be constant during filling of containers for autoclaving or the container must hold the total collection, or mixture of collections. This step is necessary to allow for testing and standardization of the bacterial polysaccharide in the final dried product.

The autoclaving process should be started immediately to prevent excess gas formation within the container that will prevent the sealing of the container. Allowing the fluid to incubate for a period of time prior to autoclaving will increase the bacterial population, however, it may also change the population (Wells, J. E. and J. B. Russell, Why Do Many Ruminal Bacteria Die and Lyse so Quickly?, 1996, J. Dairy Sci. 79:1487-1495). This probable change in population has not been studied nor the results of the resulting product tested. Although this step is contemplated and planned, until this is done, I feel that this is a major step in controlling the quality and standardization of the product.

These collections are labeled to allow control of each collected lot. Each lot collected must be tested for bacterial polysaccharides. Therefore it is important to keep each lot identified the same. In addition, each lot autoclaved must be tested for bacterial growth, both aerobic and anaerobic. It then becomes necessary that each lot autoclaved is identified, regardless of the collection lot it originated. Autoclaving should be done for a period of 45-60 minutes at of temperature of 240° F. (116° C.) and 10 pounds per square inch of pressure.

Following autoclaving, the rumen fluid collection is allowed to cool. Freeze-drying may be started immediately or it may be stored for variable amounts of time prior to further processing. Prior to freeze-drying, samples from each collection lot must be taken to be tested for bacterial polysaccharides. In addition, each lot autoclaved must be sampled and tested for bacterial growth, both aerobic and anaerobic growth. These samples must be taken prior to freeze-drying, but the testing does not have to be finished prior to freeze-drying. Each lot of freeze-dried material must be labeled with the collection lot and the autoclaved lot. The amount of material from each collection lot placed into each freeze-drying tray or lot must be recorded.

The trays are first frozen in a not frost-free freezer and then placed into the freeze-drying chamber.

### Experimental Supporting Trials

Field trials with this mixture included with the freeze dried bacterial polysaccharide resulted in improved growth rate and weight gain over the use of the bacterial polysaccharide alone. Use of the specially collected rumen fluid bacterial polysaccharide resulted in less sick animals, less mortality and fewer treatments required in calves.

### Trials

### New Mexico Calf Treatment Trial

The objective of this study was to compare 3 different treatments for calves. The main exercise here was to find if freeze-drying was an acceptable treatment for the autoclaved rumen fluid. To ensure that each treatment was randomly assigned the treatment was assigned to the calves in the order they were delivered to the calf raiser. Both bull calves and heifer calves were treated. Each calf was assigned to the treatment group according to the order of delivery to the calf raising facility, the farm of origin and the sex of the calf. Bull calves derived from other farm(s) than C_Dairy were considered a separate subgroup. Each calf was assigned to the treatment group according to the color of the treatment that was next in the rotation. The rotation was determined to be white, green and red. There were 3 subgroups in the study: C_Dairy heifers, C_Dairy bulls and other dairies' bulls. The rotation of treatments was made within each of the subgroups. For example: Two heifers are delivered on Monday. The first is assigned to the white treatment, the second is assigned to the green treatment. The first bull delivered from C_Dairy is assigned to the White treatment. The first bull from other dairies is assigned to the White treatment. On Tuesday, four more heifers are delivered. The first is assigned to the red treatment, then white, green and red. The same type of rotation was used for C_Dairy bulls and other dairies' bulls. The C_Dairy bulls were separated from the other bulls for two reasons. First there were records available from C_Dairy on dam age and colostrum administration. Second, the other bull calves were assimilated from several other dairies and owned by the calf raisers instead of C_Dairy.

The calf raisers recorded the calf's dam's number (when available) and birth date (delivery date was considered acceptable). They also recorded which treatment the calf was assigned to. If available, they were asked to check the appropriate space if the calf was a twin or if the cow had to be helped to deliver the calf (the calf was pulled). The calf should be weighed on arrival. Colored grease markers were used to mark each pen to allow the workers the ability to quickly identify the treatment group the calves are assigned to.

The treatment assigned was given for seven days. The calves were treated only 1 time per day in the morning. The calf was to receive colostrum the first day and then receive the treatment for 7 days. The medicines used for each treatment group were:

Treatment—White Calf Treatment Group—White Powder Treatment—Freeze dried autoclaved rumen fluid with probiotics, chelated trace minerals, amino acids.
Positive control—Green Calf Treatment Group—Green Liquid Treatment—Autoclaved liquid rumen fluid colored with cake coloring.
Negative Control—Red Calf Treatment Group—Red Powder Treatment—Milk powder colored with Kool-Aid®.

The mixing and feeding instructions given to the calf feeders were:

Mix the treatment in the milk prior to feeding the calf. The treatment may be mixed for several calves at once, however it may tend to settle out if allowed to stand. The bottles should be filled immediately after mixing the treatment and then inverted once or twice prior to feeding. If the milk has to stand in a five-gallon container following mixing prior to feeding or pouring into bottles, remix the container prior to pouring up for the calves. Once mixed the milk will have a color the same as the treatment group. Pink milk to the calves with a red marked pen, white milk (yellowish-gray color) to the calves with white marked pen and green milk to calves with a green marked pen.
The powder treatment is mixed at 2 level teaspoons (tsp—small spoon) per bottle. When mixing for several calves, mix ¼ cup rounded plus two tablespoons level per 5-gallon bucket.
The liquid treatment is mixed at the rate of 8 cc per bottle or 80 cc per 5-gallon bucket. Shake well before drawing out this treatment. A needle is not needed to draw it out of the bottle. The tops have slits that will allow a syringe tip to be inserted to facilitate drawing out the treatment.

The monitoring instructions used during this trial are as follows:

Although the treatment is only given for seven days, the effects are expected to last until weaning. The calves should be monitored daily until weaning. At weaning the calves should be weighed and the weight recorded on the sheet containing the calf's birth date and dam#.
Should any of the animals become sick, treat them, as is your normal practice and record the date and the medicaments used.
Daily—Record any calves that are sick, and the medicines administered.

Results: The weight gains were better for the treated animals in two of the trial groups. The group of heifers did not show the same response. The difference in the incoming weight of the three treatment groups within the heifer group may have contributed to this lack of response. The difference in the gain between the treatment group and the average of the two control groups as shown below is 6.3 #, 6.2# and 0.9# respectively. Due to irregularities in the recording of illnesses and differences in the treatments used between groups (C_Dairy vs Purchased) these data were not included into the analysis.

New Mexico Calf Trial

| Treat- | Number of Calves | In Weight | Out Weight | Gain During |
|---|---|---|---|---|
| ment | in Group | in Pounds | in Pounds | Trial |
| PBG | 17 | 88.4 | 150.6 | 61.2 |
| PBR | 15 | 93.9 | 157.9 | 64.0 |
| PBW | 17 | 90.5 | 159.4 | 68.9 |
| CBG | 17 | 90.7 | 156.8 | 66.1 |
| CBR | 17 | 89.2 | 155.4 | 66.2 |
| CBW | 17 | 88.8 | 161.2 | 72.4 |
| CHG | 17 | 77.8 | 135 | 57.2 |
| CHR | 16 | 74.8 | 134.5 | 59.7 |
| CHW | 17 | 82.0 | 141.3 | 59.3 |

P = PURCHASED
C = C_DAIRY
B = BULL CALF
H = HEIFER CALF
G = POSITIVE CONTROL with liquid product
R = NEGATIVE CONTROL
W = TREATMENT with freeze dried product

### Texas Calf Treatment Trial

The objective of this study was to compare 3 different treatments for calves. To ensure that each treatment was randomly assigned the treatment was assigned to the calves in the order they were born. Both bull calves and heifer calves were treated. Each calf was assigned to the treatment group according to the color of the card the calf's number appeared on. The cards were printed on three different color card stock. The assignment of the treatment used for each treatment group was:

Pink Calf card—Red Powder Treatment—Negative Control
White Calf card—White Powder Treatment—Warm Air Dried Positive Control
Green Calf card—Green Liquid Treatment—Treatment Group

The calf's dam's number and birth date were recorded on the cards. The workers were asked to check the appropriate space if the calf is a twin or if the cow needed assistance to deliver the calf (the calf was pulled).

The treatment assigned was given for seven days. The calves were treated only 1 time per day in the morning. The calf was to receive colostrum the first day and treatment for the next 7 consecutive days. The calf feeder was asked to circle the day of birth and then X each day the treatment is given.

The mixing and feeding instructions given to the calf feeders were:

Mix the treatment in the milk prior to feeding the calf. The treatment may be mixed for several calves at once, however it may tend to settle out if allowed to stand. The bottles should be filled immediately after mixing the treatment and then inverted once or twice prior to feeding. If the milk has to stand in a five-gallon container following mixing prior to feeding or pouring into bottles, remix the container prior to pouring up for the calves. Once mixed the milk will have a color the same as the card. Pink milk to the calves with a pink card, white milk (grayish color) to the calves with white cards and green milk to calves with a green card.
The two powder treatment are mixed as 2 level teaspoons (tsp—small spoon) per bottle. When mixing for several calves, mix 6 Tablespoons (tblsp—large spoon) per 5-gallon bucket.
The liquid treatment is mixed at the rate of 8 cc per bottle or 80 cc per 5-gallon bucket Shake well before drawing out this medicine.
Although the treatment is only given for seven days, the effects are expected to last until weaning. The calves should be monitored daily until weaning or until the individual pages are collected (this may be done prior to weaning if the calves appear normal).

The monitoring instructions used during this trial are as follows:

Daily—Record the score of the manure from the calf. The scores to be used are:
1. Normal (1)—Firm but not hard. Original form is distorted slightly after dropping to floor and settling.
2. Soft (2)—Does not hold form, piles but spreads slightly. Similar to soft serve ice cream.
3. Runny (3)—Spreads readily to about ¼ of an inch (6 mm) in depth. Similar to pancake batter.
4. Watery (4)—Liquid consistency, splatters. Similar to orange juice.
   If there is some question as to whether the manure is one score or another, for example: soft or runny, just list both scores for that day. If diarrhea develops during the day, simply write in the second score with PM after it for the later observation. If diarrhea continues for 4 days and it is watery for the four days this should be recorded each day as 4. An example of the records follows. In the example the first day (November 1) was normal and this is recorded as a 1. The second day (November 2) the calf had soft manure in the morning and watery diarrhea in the afternoon. This would be recorded as a 2 for the soft manure in the morning and as a 4 followed by PM for the watery manure in the afternoon. The next three days the calf has watery diarrhea (November 3-5, and recorded as a 4). The calf is better on November 6 and the manure is not runny but really isn't firm enough to be soft. This would be recorded as a 2 for soft and a 3 for runny. On November 7 the calf is headed for recovery and the manure is soft recorded as a 2.

| Nov 1 | Nov 2 | Nov 3 | Nov 4 | Nov 5 | Nov 6 | Nov 7 |
|---|---|---|---|---|---|---|
| 1 | 2 | 4 | 4 | 4 | 2-3 | 2 |
| | 4PM | | | | | |

Treatment descriptions are:

Green Liquid Treatment—Autoclaved liquid rumen fluid
Red Powder Treatment—Milk powder with red Kool-Aid®
White Powder Treatment—Warm Air Dried Rumen Fluid on ground rice base with added probiotics, vitamins and trace minerals.

### Results:

There were no differences in manure consistency scores between treatments. The number of antibiotic treatments administered to animals for diarrhea was reduced by 50% for treated calves. There were no deaths of treated calves but 4 and 3 deaths in the two control groups. No body weights were recorded in this trial.

Texas Calf Trial

| | # calves | # antibiotic | | # animals |
|---|---|---|---|---|
| Treat- | per | treatments | # | treated with |
| ment | treatment | for group | Deaths | antibiotics |
| GH | 12 | 9 | 0 | 6 |
| RH | 13 | 12 | 3 | 5 |
| WH | 13 | 14 | 2 | 7 |
| GB | 14 | 1 | 0 | 1 |
| RB | 13 | 8 | 1 | 5 |
| WB | 11 | 9 | 1 | 6 |
| Total G | 26 | 10 | 0 | 7 |
| Total R | 26 | 20 | 4 | 10 |
| Total W | 24 | 23 | 3 | 13 |

G = Liquid product treatment
R = Negative control
W = Heat dried positive control
B = Bull calf
H = Heifer calf
WB - Two of these calves died within 24 hours following birth

1. A method for collecting, processing, sterilizing and stabilizing rumen fluid to retain the bioactivity of the bacterial polysaccharides found therein.
